(19) United States
(12) Patent Application Publication
(10) Pub. No.: US 2026/0302690 A1
(43) Pub. Date: Oct. 1, 2026

(54) Connector For Coupling Electrode Assemblies To Current Generator

(71) Applicant: NOVOCURE GMBH, Baar (CH)

(72) Inventors: Idan Nassi, Haifa (IL); Itay Leibovich, Haifa (IL); Yoram Wasserman, Haifa (IL); Tomer Nassi, Haifa (IL)

(21) Appl. No.: 19/634,526

(22) Filed: Mar. 31, 2026

Related U.S. Application Data
(60) Provisional application No. 63/781,051, filed on Mar. 31, 2025.

(51) Int. Cl. H01R 13/62; A61N 1/40; H01R 13/703

(52) U.S. Cl. CPC H01R 13/6205; A61N 1/40; H01R 13/703

(57) ABSTRACT

An assembly includes an assembly having circuitry, the circuitry including an electrical input component that is configured to receive current from a current generator. The circuitry further includes at least one output connector port configured to magnetically couple to a respective connector along a respective axis to maintain electrical communication between the respective connector and the at least one output connector port. The electrical input component is in communication with, or configured to selectively be in communication with, the at least one output connector port. Optionally, the assembly further includes a housing having at least one flexible projection. Each flexible projection of the at least one flexible projection surrounds a respective output connector port of the at least one output connector port to form a recess defining a distal opening.

### CROSS-REFERENCE TO RELATED APPLICATIONS

This application claims priority to, and the benefit of the filing date of, U.S. Provisional Patent Application No. 63/781,051, filed Mar. 31, 2025, the entirety of which is hereby incorporated by reference herein.

### BACKGROUND

Tumor Treating Fields (TTFields) therapy is a proven approach for treating tumors using alternating electric fields at frequencies between 50 KHz – 1 MHz, such as 100-500 kHz. Conventionally, the alternating electric fields are induced by electrode assemblies (e.g., arrays of capacitively coupled electrodes, also called transducer arrays) placed on opposite sides of a target location in the subject’s body. When an AC voltage is applied between opposing electrode assemblies, an AC current is coupled through the electrode assemblies and into the subject’s body. And higher currents are strongly correlated with higher efficacy of treatment.

The electrode assembly or assemblies for delivering treatment can be connected to a signal generator through a connector. The electrode assemblies can have respective connector units. Various difficulties arise through use of such connector units, including inadvertent decoupling and inflexible couplings leading to twisted wires or cables.

### SUMMARY

Disclosed herein, in various aspects, is an assembly comprising circuitry, the circuitry comprising an electrical input component that is configured to receive current from a current generator. The circuitry further comprises at least one output connector port. Each output port of the at least one output port is configured to magnetically couple to a respective connector along a respective axis to maintain electrical communication between the respective connector and said output connector port. The electrical input component is in communication with, or configured to selectively be in communication with, the at least one output connector port. The assembly further comprises a housing comprising at least one flexible projection. Each flexible projection of the at least one flexible projection surrounds a respective output connector port of the at least one output connector port to form a recess defining a distal opening.

Also disclosed herein is a system comprising an assembly that includes circuitry comprising an electrical input component that is configured to receive current from a current generator. The circuitry further comprises at least one output connector port. Each output port of the at least one output port is configured to magnetically couple to a respective connector along a respective axis to maintain electrical communication between the respective connector and said output connector port. The electrical input component is in communication with, or configured to selectively be in communication with, the at least one output connector port. The assembly further comprises a housing comprising at least one flexible projection. Each flexible projection of the at least one flexible projection surrounds a respective output connector port of the at least one output connector port to form a recess defining a distal opening. The system further comprises a cable comprising the respective connector that is configured to couple to the at least one output connector port.

### BRIEF DESCRIPTION OF THE DRAWINGS

FIG. 1 is a perspective view of an assembly comprising a connector unit having a plurality of output connector ports and a connector configured to couple to one of the output connector ports.

FIG. 2 is a perspective view of the assembly of FIG. 1 with a portion of a housing of the connector unit omitted to show underlying elements.

FIG. 3 is a perspective view of an exemplary output connector port of FIG. 1 and a respective connector configured to couple to the output connector port.

FIG. 4 is a schematic cross-sectional diagram showing flexing of a flexible projection of the housing of the connector unit to decouple a respective connector from an output connector port.

FIG. 5 is a block diagram of a system for providing treatment comprising the assembly as in FIG. 1.

Various embodiments are described in detail below with reference to the accompanying drawings, wherein like reference numerals represent like elements.

### DETAILED DESCRIPTION

This application describes exemplary electrode assemblies that can be used, e.g., for delivering TTFields to a subject’s body and treating one or more cancers or tumors located in the subject’s body.

The present invention can be understood more readily by reference to the following detailed description, examples, drawings, and claims, and their previous and following description. However, it is to be understood that this invention is not limited to the specific apparatuses, devices, systems, and/or methods disclosed unless otherwise specified, and as such, of course, can vary.

Headings are provided for convenience only and are not to be construed to limit the invention in any manner. Embodiments illustrated under any heading or in any portion of the disclosure may be combined with embodiments illustrated under the same or any other heading or other portion of the disclosure.

Any combination of the elements described herein in all possible variations thereof is encompassed by the invention unless otherwise indicated herein or otherwise clearly contradicted by context.

As used in the specification and the appended claims, the singular forms “a,” “an” and “the” include plural referents unless the context clearly dictates otherwise. Thus, it is contemplated that disclosure of a singular form of an element can provide support for embodiments in which only a single such element is provided, as well as support for embodiments in which a plurality of such elements are provided.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Optionally, in some aspects, when values are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

Conventionally, a TTFields signal is conveyed from an AC Voltage (Current) Generator along a single cable. The TTFields signal from the single cable is often divided between a plurality of transducer arrays that are provided in optimized locations on the body or on the head for delivery of TTFields. For example, two pairs of transducer arrays can be used. Each array of the pair can be positioned on opposing sides of the target. The pairs of arrays can be positioned relative to each other to produce two orthogonal electric field directions. A connector (e.g., a 1-to-4 connector) can be used to divide the TTFields signal from the single cable to each transducer array of the two pairs of transducer arrays. The subject may resume daily activities while receiving treatment. Occasionally, the cable to a given array may get caught or pulled, thereby disconnecting the cable from the connector. Occasionally, this may occur with a minor shock or electrical stimulation. However, presenting a more solid connection (e.g., a connection that resists inadvertent decoupling) has presented difficulties for patients both in initial set up, and when deliberately connecting or disconnecting the cable (connector pins are bent or broken due to an undue amount or direction of force). The present disclosure provides assemblies and systems capable of delivering stable TTFields signals (for example, 1-10 Amps alternating current waveform having a frequency of from 50 kHz to 1 MHz) and having connection points with an easy disconnect for deliberate disconnections, while inhibiting accidental disconnections. Moreover, any unwanted disconnection is configured to cause a current interrupt mechanism that prevents the aforementioned unwanted minor shock or electrical stimulation.

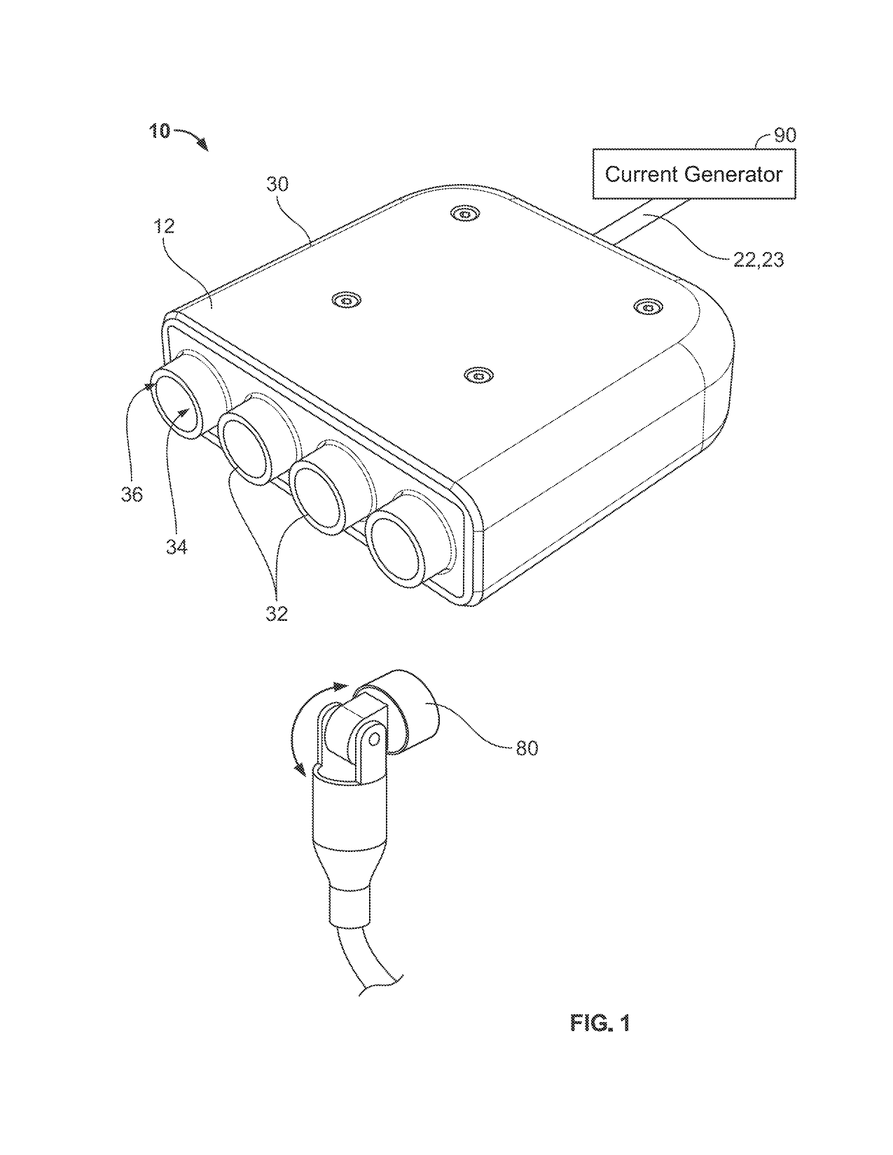

Disclosed herein, and with reference to FIGS. 1-3, is an assembly 10 comprising circuitry 20, the circuitry comprising an electrical input component 22 that is configured to receive current from a current generator 90 (e.g., a signal generator) and at least one output connector port 24 configured to couple to a respective connector 80 along a respective axis 26 to maintain electrical communication between the respective connector and the at least one output connector port. The electrical input component 22 can be in electrical communication with, or configured to selectively be in electrical communication with, the at least one output connector port 24. In some exemplary aspects, the at least one output connector port can comprise or consist of a single output connector port. In other exemplary aspects, the at least one output connector port can comprise a plurality of output connector ports.

As shown in FIG. 1, the assembly 10 can further comprise a housing 30 comprising at least one flexible projection 32 (e.g., a plurality of flexible projections). Each flexible projection 32 of the at least one flexible projection can surround a respective output connector port 24 of the at least one output connector port to form a recess 34 defining a distal opening 36. Each output connector port 24 of the at least one output connector port 24 can be configured to magnetically couple to a respective connector 80. For example, one or both of the at least one output connector port 24 and the respective connector 80 can comprise a magnet 44 (FIG. 4). Optionally, one of the at least one output connector port 24 or the respective connector 80 can comprise a magnet 44, and the other can comprise a material that is magnetizable (configured to be magnetized) in the presence of a magnet (magnetizable material 46) (such that the magnetic materials of the output connector port 24 and the respective connector 80 are magnetically attracted to one another). For example, the at least one output connector port 24 can comprise a magnet 44. In some aspects, the respective connector 80 can comprise a magnet. Optionally, in these aspects, the at least one output connector port 24 can comprise a magnetic material 46 that is magnetizable in the presence of the magnet of the respective connector 80 (such that the magnetic material of the connector port 24 is magnetically attracted to and coupled to the magnet of the respective connector 80, or vice-vera: the magnetic material of the connector 80 is magnetically attracted to and coupled to the magnet of the respective connector port 24).

Each flexible projection 32 can be configured to inhibit decoupling between the at least one output connector port 24 and the respective connector 80. For example, the at least one output connector port 24 and the respective connector 80 can have a magnetic coupling sufficient to greatly inhibit decoupling by movement of the respective connector parallel to or in alignment with the respective axis 26. Thus, the assembly 10 can be configured such that movement of the respective connector 80 in a direction parallel to or in alignment with the respective axis 26 is not sufficient to decouple the magnetic materials of the respective connector and the output connector port 24. Rather, the magnetic coupling can be more easily overcome by movement of the respective connector 80 transverse to or substantially transverse to the respective axis 26 or, referring to FIG. 4, pivoting the respective connector 80 relative to the at least one output connector port 24 to separate a first side 82a while a second side 82b remains in contact with the at least one output connector port, using leverage of the second side 82b to separate the magnet/magnetic materials of the at least one output connector port 24 and the respective connector 80. Each flexible projection 32 can inhibit such transverse or pivotal movement of the respective connector 80. In this way, the flexible projection(s) 32 can inhibit inadvertent decoupling that can lead to electrical stimulation that causes physical sensation. Referring also to FIG. 1, in some aspects, the connector 80 can have an electrical coupling end portion that is pivotable relative to a cable end portion of the connector about an axis that is transverse to (e.g., perpendicular to) the respective axis 26. In some aspects, the electrical coupling end portion can be configured to pivot by at least 45 degrees, or at least 90 degrees, or at least 170 degrees, or at least 180 degrees relative to the cable end portion of the connector 80.

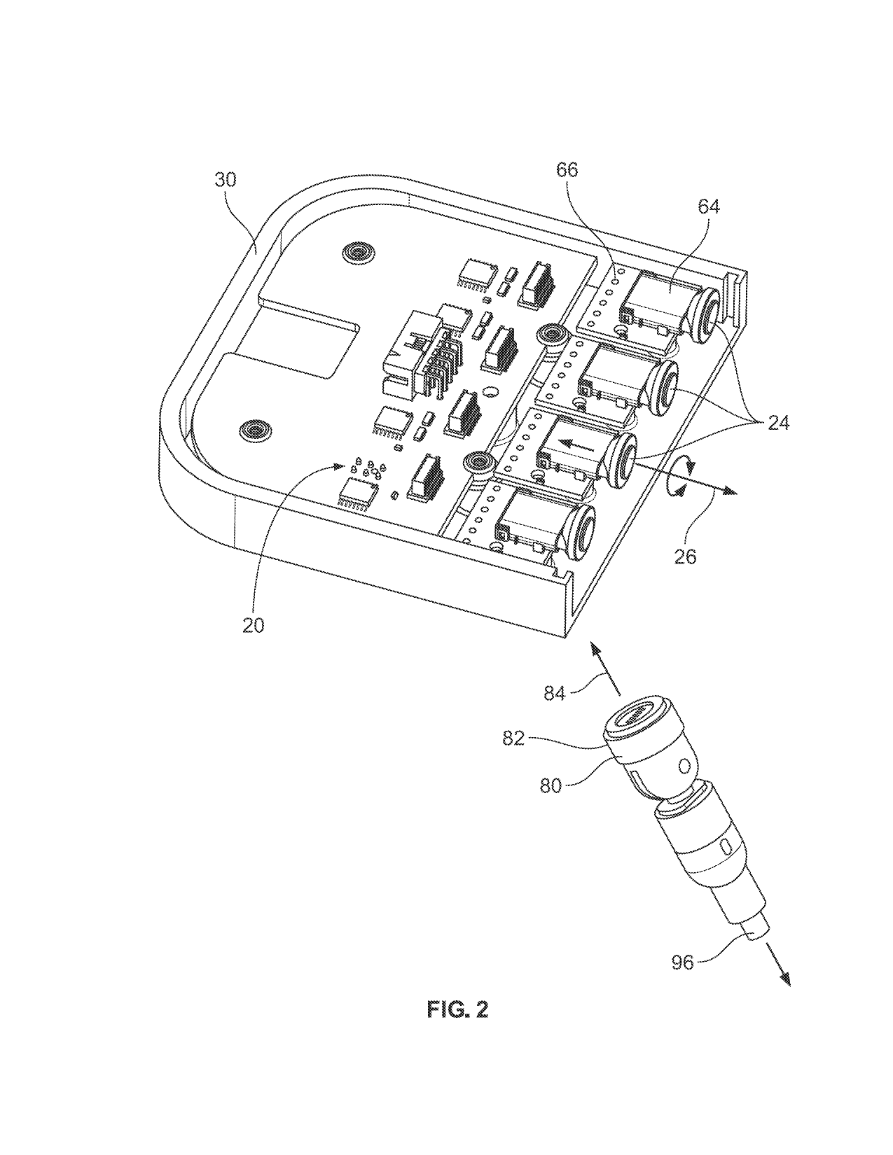

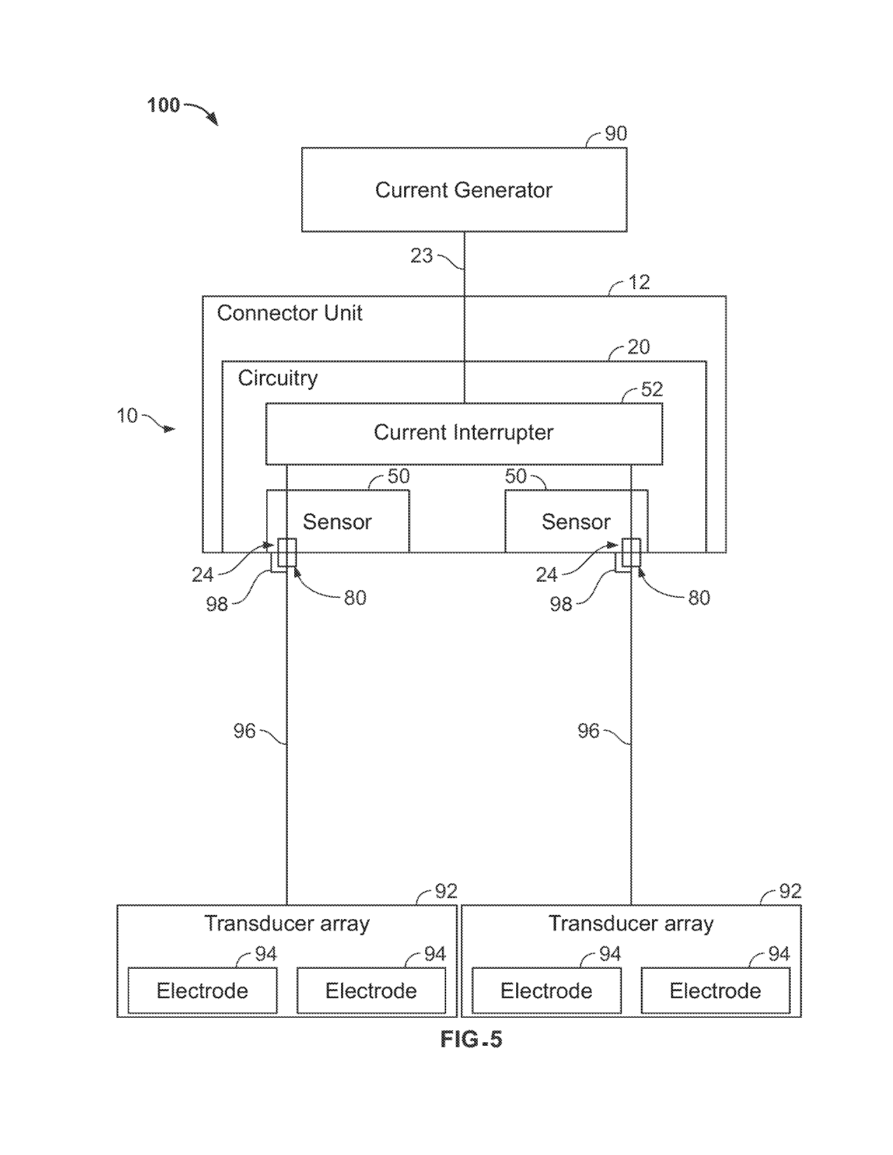

As shown in FIG. 2, the circuitry 20 can be at least partly received within the housing 30. In various aspects, the housing 30 and the circuitry 20 can be provided as a connector unit 12. In further aspects, the at least one output port 24 can comprise a plurality of output ports. Each output port 24 of the plurality of output ports can electrically couple to a transducer array 92, with each transducer array comprising at least one electrode element 94 (FIG. 5). Accordingly, the connector unit 12 can be configured to distribute current from the current generator 90 to a plurality of transducer arrays 92. Further, in some aspects, each output port 24 of the at least one output port (optionally, plurality of output ports) can be on a respective channel. In this way, the current generator 90 and the connector unit 12 can cooperate to distribute current to the plurality of transducer arrays 92 to thereby generate electric fields by the plurality of transducer arrays 92. In other optional aspects, the connector unit 12 can have only a single output port 24.

In various aspects, the electrical input component 22 can comprise a main cable 23. Optionally, in these aspects, the main cable 23 can be integrally coupled (e.g., non-removably coupled) to the housing 30. In other aspects, the electrical input component 22 can comprise a main cable connector (e.g., a socket) that is configured to electrically couple to a main cable (e.g., a plug of the main cable that is receivable within the socket of the assembly 10).

The at least one output connector port 24 can be configured to maintain electrical communication between the respective connector 80 and the at least one output connector port while permitting the respective connector to rotate about the respective axis 26 relative to the at least one output connector port. For example, each output connector port 24 of the at least one output connector port 24 can comprise a plurality of electrical leads 40, wherein each electrical lead of the plurality of electrical leads is an annulus or a segment of an annulus. The respective connector 80 can comprise a plurality of spring pins 42 that are each configured to contact a respective electrical lead 40. In other aspects, each output connector port 24 of the at least one output connector port 24 can comprise a plurality of spring pins, and the respective connector 80 can comprise a plurality of electrical leads. For example, each electrical lead 40 of the plurality of electrical leads can be an annulus or a segment of an annulus, and each spring pin 42 of the respective connector 80 can be configured to electrically contact a respective electrical lead. For example, each annulus can be centered around the respective axis 26, and each spring pin 42 can be radially spaced from the respective axis 26 in order to contact a respective annulus to establish electrical communication.

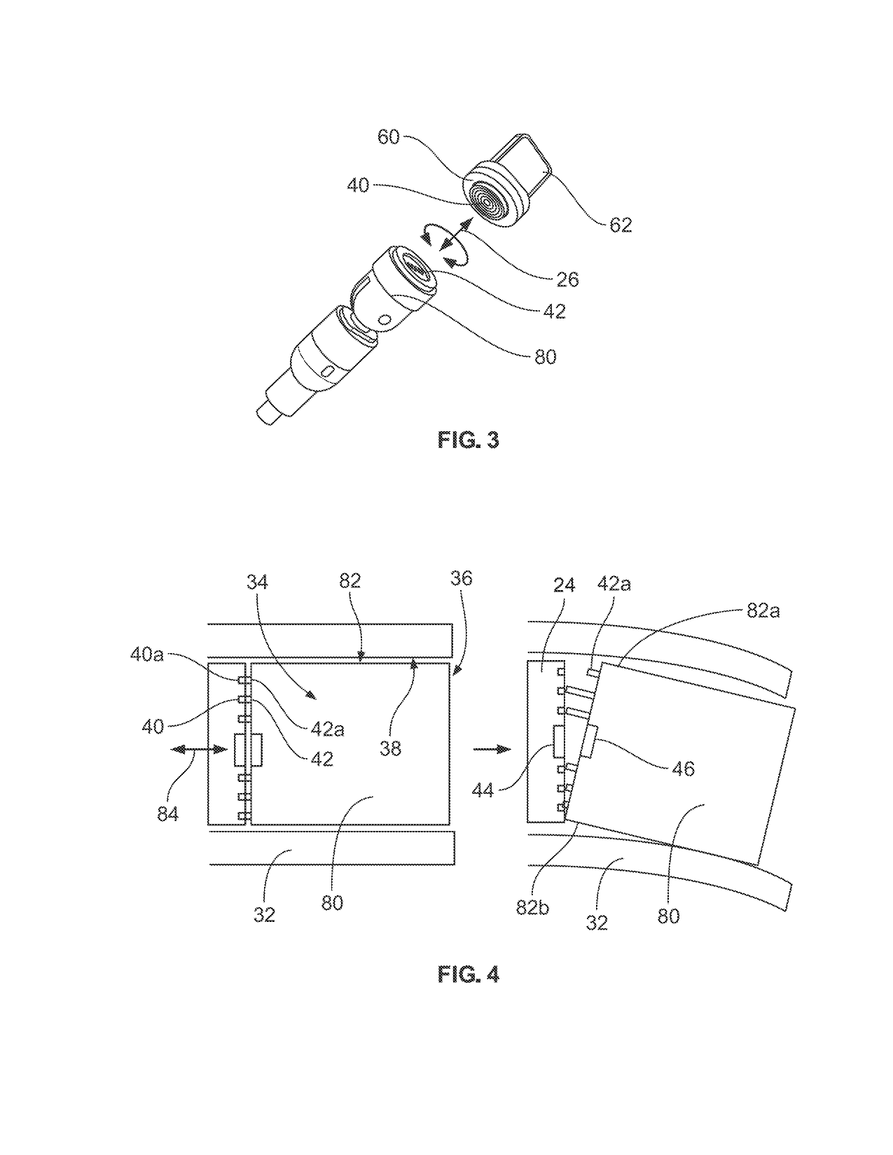

Optionally, each output connector port 24 of the at least one output connector port 24 can be configured to maintain electrical communication with the respective connector 80 while also permitting the respective connector to rotate about the respective axis 26 relative to the at least one output connector port (see FIG. 3) by an amount ranging from 0 degrees to 180 degrees, or from 0 degrees to at least 180 degrees, or from 40 degrees to 180 degrees, or from 180 degrees to 360 degrees, or from 0 degrees to 360 degrees (e.g., permitting full revolutions while maintaining electrical communication).

In some aspects, the recess 34 of each flexible projection 32 can have a depth from 3 mm to 10 mm, wherein the depth corresponds to a distance from the distal opening 36 to the respective output connector port 24 surrounded by said flexible projection 32. For example, the depth can be from about 3 mm to about 6 mm, from about 4 mm to about 1 cm, from about 4 mm to about 6 mm, from about 4 mm to about 8 mm, from about 6 mm to about 8 mm, or from about 6 mm to about 10 mm, or from about 8 mm to about 10 mm, from about 5 mm to about 15 mm, or from about 5 mm to about 9 mm, or from about 5 mm to about 8 mm.

In various aspects, the at least one flexible projection 32 can have a Shore 00 durometer from 80 to 100. For example, the at least one flexible projection 32 can have a Shore 00 durometer from 85 to 95, or about 90. In exemplary aspects, the flexible projection 32 can comprise or consist of silicone, rubber, flexible polyvinyl chloride (PVC), or thermoplastic polyurethane (TPU).

Referring to FIG. 4, the length and the flexibility/rigidity of the at least one flexible projection 32 can cooperate to provide a resistance to deflection. The resistance to deflection can be predetermined to inhibit inadvertent decoupling (through accidental shear) of the respective connector 80 from an output connector port 24 of the at least one output connector port 24 while permitting a user to decouple the respective connector when desired. For example, the at least one flexible projection 32 can be configured to engage an outer surface 82 of the respective connector 80, to resist (a) shear of the respective connector 80 and (b) deflection of a central axis 84 of the respective connector 80 from the respective axis 26 of the at least one output connector port 24.

In some aspects, and with reference to FIG. 4, the at least one flexible projection 32 can have a circumferential clearance between an inner wall 38 defining the recess 34 and the outer surface 82 of the respective connector 80. The clearance can correspond to an amount of angular deflection of the central axis 84 of the respective connector 80 from the respective axis 26 of the at least one output connector port 24 prior to contact between the respective connector 80 and the inner wall 38 of the at least one flexible projection 32. In some aspects, the clearance can be zero. In other aspects, the clearance can be less than ½ mm, or less than 1 mm on each side. In some optional aspects, the recess 34 of the at least one flexible projection 32 can be cylindrical. In other aspects, the recess 34 can have polygonal cross sections, or any suitable geometry to engage the respective connector 80 as described herein. Optionally, the recess 34 can taper proximally, toward the at least one output connector port 24. In other optional aspects, the recess 34 can have consistent cross sectional profiles and areas along the respective axis 26.

In some optional aspects, each output connector port 24 can have a first end 60 that is configured to couple to the respective connector 80 and a second end 62 that is receivable into a socket 64, such as a socket of a circuit board 66. Optionally, the second end 62 of the output connector port 24 and the socket 64 can comprise male and female USB-C connectors, respectively.

In further aspects, and with reference to FIG. 5, the circuitry 20 of the assembly 10 can comprise a sensor 50 that is configured to detect a decoupling between the respective connector 80 and the at least one output connector port 24. The circuitry 20 can further comprise a current interrupter 52 that is configured to interrupt current between the current generator 90 and the at least one output connector port 24 upon the sensor detecting the decoupling. This can inhibit physical sensation of electrical stimulation associated with inadvertent decoupling. The current interrupter 52 can comprise, for example, a relay or other switch, or an analog to digital converter coupled to an interface of a microprocessor, that stops (or significantly reduces) electrical communication between the current generator 90 and the at least one output connector port 24. In this way, the assembly 10 can be configured to interrupt current between the current generator 90 and the at least one output connector port 24. The current interrupter 52 can be provided at any location between the current generator 90 and the at least one output connector port 24. Accordingly, the current interrupter 52 need not be provided in the housing 30, although it may. Optionally, each output connector port 24 of the at least one output connector port can comprise, or be associated with, a respective sensor 50. In other aspects, two or more (optionally, all output connector ports of the at least one output connector port 24) can couple to the same sensor 50. Further, in some aspects, each output connector port 24 of the at least one output connector port can comprise, or be associated with, a respective current interrupter 52. In other aspects, two or more (optionally, all output connector ports of the at least one output connector port 24) can couple to the same current interrupter 52.

Referring to FIGS. 4 and 5, in some aspects, the sensor 50 can be configured to detect current or voltage through an outermost electrical lead 40a of the plurality of electrical leads 40. It is contemplated that outermost electrical leads (e.g., the electrical leads positioned at the most radial outward location within the output connector port) can be most likely to be decoupled first. Accordingly, at least one electrical lead 40 of the plurality of electrical leads located radially inward of the outermost electrical lead can be configured to conduct current to generate the tumor treating fields. In this way, the interrupter 52 can interrupt current between the current generator 90 and the at least one output connector port 24 before inner electrical leads that are conducting current to the transducer array(s) 92 become decoupled. Accordingly, the radially outermost spring pin or pins 42 of the connector 80 can serve as an indicator electrical connector or connectors. The sensor 50 can comprise a current or voltage sensor that is in electrical communication with the outermost electrical lead 40a. The sensor 50 can sense a change in current at the electrical lead 40a, thereby detecting decoupling of the connector 80 from the output connector port 24 before the inner electrical leads disconnect.

In still further aspects, the indicator electrical connector or connectors 42a (e.g., radially outermost spring pin or pins 42) can have a shorter protrusion length than the other electrical connectors (e.g., the rest of the spring pins 42). In this way, the indicator electrical connector or connectors can be configured to decouple first from the output connector port 24 as the connector 80 is being moved from the output connector port. In this way, a change in current sensed by the sensor in electrical communication with the outermost electrical lead 40a can indicate that the connector 80 is being moved from the output connector port.

In some aspects, the sensor 50 can comprise at least one pressure sensor. Each pressure sensor can be configured to detect a pressure between a respective output connector port of the at least one output connector port 24 and the respective connector 80. Accordingly, a reduced pressure can be indicative of separation between the at least one output connector port 24 and the respective connector 80. The interrupter 52 can be configured to interrupt current upon feedback from the sensor. For example, the interrupter 52 can interrupt current when a pressure/force sensed is below a predetermined threshold.

In some aspects, the sensor 50 can comprise at least one magnetic field strength sensor. Each magnetic field strength sensor can be configured to detect a magnetic field strength between a respective output connector port 24 and the respective connector 80. For example, the sensor 50 can comprise a Hall Effect sensor or a magnetoresistive sensor. Accordingly, based on a decrease in magnetic field strength as measured by the sensor, the sensor 50 can detect movement of the respective connector 80 from the output connector port 24, and the current interrupter 52 can, based on the feedback from the magnetic sensor, interrupt the current. Optionally, the current interrupter 52 can interrupt current based on the magnetic field sensed being below a predetermined threshold.

Additional embodiments of configurations for providing current interruption are disclosed in U.S. Patent No. 12,083,332, granted September 10, 2024 and U.S. Patent Application No. 18/898,993, filed September 27, 2024, the entirety of each of which is hereby incorporated by reference herein.

In some optional aspects, the assembly 10 can comprise a lock 98 that is configured to inhibit decoupling of the respective connector 80 from the at least one output connector port 24. The lock 98 can comprise, for example, a strap, a cage, a detent, a lever, a hook, or any suitable structure that engages the respective connector 80 to inhibit unintentional detachment of the respective connector 80 from the at least one output connector port 24. Optionally, the lock 98 can comprise a release that disengages the lock 98 to permit decoupling of the respective connector 80. The release can comprise a button, lever, pin, spring, or other suitable structure.

In some aspects, each output connector port 24 of the at least one output connector port can be configured to provide at least 4 electrical connections. In some aspects, each output connector port 24 of the at least one output connector port can be configured to conduct at least 9 amps.

In some aspects, the electrical input component 22 can be permanently in electrical communication with the at least one output connector port 24. In other aspects, the electrical input component 22 can be configured to selectively be in electrical communication with the at least one output connector port 24. Accordingly, in some optional aspects, the circuitry can comprise switching that selectively provides (e.g., selectively establishes and breaks) electrical communication.

Referring to FIG. 5, a system 100 can comprise the assembly 10 and a cable 96, the cable comprising the respective connector 80 that is configured to couple to the at least one output connector port 24 of the circuitry of the assembly 10. The system 100 can further comprise a transducer array 92 comprising at least one electrode element 94. The cable 96 can be electrically coupled to the at least one electrode element 94 of the transducer array 92. Optionally, the system 100 can comprise a plurality of transducer arrays 92, each comprising one or a plurality of electrodes elements 94. A respective cable 96 can be electrically coupled between the electrode element(s) 94 of each transducer array 92 and a respective output connector port 24 of the circuitry 20 of the connector unit 12. The system 100 can further comprise a current generator 90 configured to cooperate with the transducer array (optionally, a plurality of transducer arrays) to generate tumor treating fields.

The current generator 90 may generate one or more electric signals in the shape of waveforms or trains of pulses. The current generator 90 may be configured to generate an alternating voltage waveform at frequencies in the range from about 50 KHz to about 1 MHz (preferably from about 50 KHz to about 500 KHz or from about 100 KHz to about 300 KHz) (e.g., the TTFields). The voltages are such that the electrical field intensity in tissue to be treated is typically in the range of about 0.1 V/cm to about 10 V/cm.

The current generator 90 may be coupled, via the assembly 10, to one or more transducer arrays 92 that are activated by the electric signals (e.g., waveforms). The cables 96 can comprise conductive leads that include standard isolated conductors with a flexible metal shield and may be grounded to prevent the spread of the electrical field generated by the conductive leads. The current generator 90 can provide outputs to the transducer arrays that can be operated simultaneously or sequentially. Output parameters of the current generator 90 may comprise, for example, an intensity of the field, a frequency of the waves (e.g., treatment frequency), and a maximum allowable temperature of the one or more stimulation zones (e.g., transducer arrays 92). The output parameters may be set and/or determined by control software in conjunction with a processor of the current generator. After determining a desired (e.g., optimal) treatment frequency, the control software may cause the processor to send a control signal to cause the current generator to output the desired treatment frequency to the transducer array(s) 92. Similarly, it is contemplated that the processor can be in electrical communication with one or more temperature sensor (e.g., a thermistor or thermocouple) that is configured to measure temperature at a respective transducer array, and when a temperature threshold is reached, the control software can cause the processor to decrease a frequency and/or intensity of the electric signal provided by the current generator. In further aspects, it is contemplated that the processor can be in electrical communication with a sensor that is configured to measure an intensity of the electric field generated by the transducer array(s) 92, and the control software can cause the processor to decrease or increase a frequency and/or intensity of the electric signal to achieve a desired decrease or increase in the field intensity. In example systems as described herein, the system can deliver TTFields signals (e.g., with a current of 2A or 4A or between 2A and 4A) over a period of several hours with stable TTFields signal current and frequency, and a stable temperature (e.g., from 25℃ to 27℃ ). The disclosed connectors have been confirmed to be capable of providing such TTFields signals with such stable current, frequency, and temperature.

The one or more transducer array(s) 92 may be configured in a variety of shapes and positions so as to generate an electrical field of the desired configuration, direction and intensity at a target volume so as to focus treatment. Optionally, the transducer array(s) 92 may be configured to deliver two perpendicular field directions through a volume of interest (e.g., a target region).

The connectors described herein are used to couple one or more electrode assemblies (e.g., transducer arrays 92) to the current generator 90. In some aspects, and as illustrated in FIG. 1, the at least one output connector port 24 can be provided as a component that is independent of the current generator and the transducer array 92. In further aspects, the current generator 90 can be directly connected with magnetic connectors at either or both ends of the cable. For example, such a connector can be provided at, or adjacent to, the generator or at, or adjacent to, the transducer arrays), or as described herein, connected via a housing 30 between the current generator 90 and the transducer arrays 92, with the connection occurring at the housing. Accordingly, in some aspects, an output connector port 24 as disclosed herein can be provided at (e.g., integral to) the current generator 90, a transducer array 92, and/or at an intervening component (e.g., at the housing 30).

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: An assembly comprising:

circuitry comprising:

an electrical input component that is configured to receive current from a current generator;

at least one output connector port, each output connector port of the at least one output connector port configured to magnetically couple to a respective connector such that the output connector port and the respective connector are aligned along a respective axis to maintain electrical communication between the respective connector and the at least one output connector port;

wherein the electrical input component is in electrical communication with, or configured to selectively be in electrical communication with, the at least one output connector port;

a housing comprising at least one flexible projection, wherein each flexible projection of the at least one flexible projection surrounds a respective output connector port of the at least one output connector port to form a recess defining a distal opening.

Aspect 2: The assembly of aspect 1, wherein the at least one output connector port comprises a magnet.

Aspect 3: The assembly of aspect 1, wherein the at least one output connector port comprises a magnetic material that is magnetizable in the presence of a magnet of the respective connector.

Aspect 4: The assembly of any one of the preceding aspects, wherein the at least one output connector port comprises one of:

a plurality of spring pins; or

a plurality of electrical leads, wherein each electrical lead of the plurality of electrical leads is an annulus or a segment of an annulus.

Aspect 5: The assembly of aspect 4, wherein the at least one output connector port comprises a plurality of electrical leads, wherein each electrical lead of the plurality of electrical leads is an annulus or a segment of an annulus.

Aspect 6: The assembly of any one of the preceding aspects, wherein the at least one output connector port is configured to maintain electrical communication between the respective connector and the at least one output connector port while permitting the respective connector to rotate about the respective axis relative to the at least one output connector port by an amount ranging from 0 degrees to at least 180 degrees, or up to 360 degrees.

Aspect 7: The assembly of any one of the preceding aspects, wherein the at least one output connector port is configured to maintain electrical communication between the respective connector and the at least one output connector port while permitting the respective connector to pivot about the respective axis by an amount ranging from 0 degrees to at least 180 degrees relative to the at least one output connector port.

Aspect 8: The assembly of any one of the preceding aspects, wherein the recess of the at least one flexible projection is cylindrical.

Aspect 9: The assembly of any one of the preceding aspects, wherein the recess of each flexible projection of the at least one flexible projection has a depth from 4 mm to 10 mm, wherein the depth corresponds to a distance from the distal opening to the respective output connector port surrounded by said flexible projection.

Aspect 10: The assembly of any one of the preceding aspects, wherein the at least one flexible projection has a Shore 00 durometer from 80 to 100.

Aspect 11: The assembly of any one of the preceding aspects, wherein the circuitry further comprises:

a sensor that is configured to detect a decoupling between the respective connector and the at least one output connector port; and

a current interrupter that is configured to interrupt current between the current generator and the at least one output connector port upon the sensor detecting the decoupling.

Aspect 12: The assembly of aspect 11, wherein the at least one output connector port comprises a plurality of electrical leads, wherein each electrical lead of the plurality of electrical leads is an annulus or a segment of an annulus, and wherein the sensor is configured to detect current or voltage through an outermost electrical lead of the plurality of electrical leads.

Aspect 13: The assembly of aspect 11, wherein the sensor comprises at least one pressure sensor, wherein each pressure sensor is configured to detect a pressure between a respective output connector port of the at least one output connector port and the respective connector.

Aspect 14: The assembly of aspect 11, wherein the sensor comprises at least one magnetic field strength sensor, wherein each magnetic field strength sensor is configured to detect a magnetic field strength between a respective output connector port of the at least one output connector port and the respective connector.

Aspect 15: The assembly of any one of the preceding aspects, further comprising a lock that is configured to inhibit decoupling of the respective connector from the at least one output connector port.

Aspect 16: The assembly of any one of the preceding aspects, wherein each one output connector port of the at least one output connector port is configured to provide at least 4 electrical connections.

Aspect 17: The assembly of any one of the preceding aspects, wherein each one output connector port of the at least one output connector port is configured to conduct at least 9 amps.

Aspect 18: The assembly of any one of the preceding aspects, wherein the at least one output connector port comprises a plurality of output ports.

Aspect 19: The assembly of any one of the preceding aspects, wherein the electrical input component comprises a main cable integrally coupled to the housing.

Aspect 20: The assembly of any one of the preceding aspects, wherein the electrical input component comprises a main cable integrally coupled to the housing and wherein the current generator is configured to supply the main cable with an alternating current waveform at a frequency in a range from 50 kHz to 1 MHz.

Aspect 21: The assembly of any one of aspects 1-18, wherein the electrical input component comprises a main cable connector that is configured to electrically couple to a main cable.

Aspect 22: The assembly of any one of aspects 1-18, wherein the electrical input component comprises a main cable connector that is configured to electrically couple to a main cable and wherein the current generator is configured to supply the main cable with an alternating current waveform at a frequency in a range from 50 kHz to 1 MHz.

Aspect 23: The assembly of any one of the preceding aspects, wherein each output connector port of the at least one output connector port comprises either:

a magnet; or

a magnetic material that is magnetizable in the presence of a magnet of the respective connector.

Aspect 24: A system comprising:

an assembly that includes:

circuitry comprising:

an electrical input component that is configured to receive current from a current generator;

at least one output connector port, each output connector port of the at least one output connector port configured to magnetically couple to a respective connector such that the output connector port and the respective connector are aligned along a respective axis to maintain electrical communication between the respective connector and the at least one output connector port;

wherein the electrical input component is in electrical communication with, or configured to selectively be in electrical communication with, the at least one output connector port;

a housing comprising at least one flexible projection, wherein each flexible projection of the at least one flexible projection surrounds a respective output connector port of the at least one output connector port to form a recess defining a distal opening; and

a cable comprising the respective connector that is configured to couple to the at least one output connector port.

Aspect 25: The system of aspect 24, further comprising a transducer array comprising at least one electrode element, wherein the cable is electrically coupled to the at least one electrode element of the transducer array.

Aspect 26: The system of aspect 25, further comprising a current generator configured to cooperate with the transducer array to generate tumor treating fields.

Aspect 27: The system of aspect 26, wherein the current generator is configured to cooperate with the transducer array to the generate tumor treating fields at a frequency in a range from 50 kHz to 1 MHz.

Aspect 28: The system of aspect 27, wherein the at least one output connector port comprises one of:

a plurality of spring pins; or

a plurality of electrical leads, wherein each electrical lead of the plurality of electrical leads is an annulus or a segment of an annulus,

wherein the respective connector of the cable comprises the other of a plurality of spring pins; or

a plurality of electrical leads, wherein each electrical lead of the plurality of electrical leads is an annulus or a segment of an annulus,

wherein an outermost electrical lead of the plurality of electrical leads and/or an outermost spring pin aligned to contact the outermost electrical lead is in electrical communication with a current interrupter that is configured to cease generation of tumor treating fields upon decoupling of a respective spring pin of the plurality of spring pins.

Aspect 29: The system of aspect 28, wherein at least one electrical lead of the plurality of electrical leads located radially inward of the outermost electrical lead is configured to conduct current to generate the tumor treating fields.

Aspect 30: The assembly of any one of aspects 24-29, wherein the at least one output connector port comprises a magnet.

Aspect 31: The assembly of any one of aspects 24-30, wherein the respective connector of the cable comprises a magnet.

Aspect 32: The system of any one of aspects 24-31, wherein the respective connector is receivable within the recess of the at least one flexible projection with a clearance of no more than 1 mm on each side.

Aspect 33: The system of any one of aspects 24-32, wherein the at least one output connector port comprises a plurality of electrical leads, wherein the plurality of electrical leads are annuluses or segments of annuluses, wherein the circuitry of the assembly further comprises:

a sensor that is configured to detect a decoupling between the respective connector and the at least one output connector port by detecting a current or voltage through an outermost electrical lead of the plurality of electrical leads; and

a current interrupter that is configured to interrupt current at any point between the current generator and the at least one output connector port upon the sensor detecting the decoupling,

wherein the respective connector of the cable comprises a plurality of spring pins, wherein each spring pin of the plurality of spring pins is configured to project outwardly by a respective maximum distance, wherein a radially outermost spring pin of the plurality of spring pins is configured to project outwardly by a first maximum distance that is less than the respective maximum distance of at least one other spring pin of the plurality of spring pins.

Aspect 34: The system of any one of aspects 24-33, wherein the respective connector comprises at least one indicator electrical connector and a plurality of conductive elements, wherein the apparatus further comprises:

at least one associated monitoring circuit configured to provide feedback related to a status of a connection between the respective connector and the at least one output connector port, the at least one associated monitoring circuit connected to the at least one indicator electrical connector configured to disconnect from the at least one output connector port at a first position relative to the at least one output connector port and wherein the first plurality of conductive elements is configured to disconnect from the at least one output connector port at a second position relative to the at least one output connector port, the second position being different from the first position.

Aspect 35: A method comprising:

generating tumor treating fields using:

the system as in any one of aspects 24-34;

a transducer array comprising at least one electrode element, wherein the cable is electrically coupled to the at least one electrode element of the transducer array; and

a current generator configured to cooperate with the transducer array to generate tumor treating fields.

Aspect 36: The method of aspect 35, wherein the tumor treating fields have a frequency in a range from 50 kHz to 1 MHz.

Aspect 37: An assembly comprising:

circuitry comprising:

an electrical input component that is configured to receive current from a current generator;

at least one output connector port, each output connector port of the at least one output connector port configured to magnetically couple to a respective connector such that the output connector port and the respective connector are aligned along a respective axis to maintain electrical communication between the respective connector and the at least one output connector port while permitting the respective connector to rotate and/or pivot about the respective axis relative to the at least one output connector port;

wherein the electrical input component is in electrical communication with, or configured to selectively be in electrical communication with, the at least one output connector port;

a housing comprising at least one flexible projection, wherein each flexible projection of the at least one flexible projection surrounds a respective output connector port of the at least one output connector port to form a recess defining a distal opening.

Aspect 38: The assembly of aspect 37, wherein the at least one output connector port comprises one of:

a plurality of spring pins; or

a plurality of electrical leads, wherein each electrical lead of the plurality of electrical leads is an annulus or a segment of an annulus.

Aspect 39: The assembly of aspect 38, wherein the at least one output connector port comprises a plurality of electrical leads, wherein each electrical lead of the plurality of electrical leads is an annulus or a segment of an annulus.

Aspect 40: The assembly of any one of aspects 37-39, wherein the at least one output connector port is configured to maintain electrical communication between the respective connector and the at least one output connector port while permitting the respective connector to rotate about the respective axis relative to the at least one output connector port by an amount ranging from 0 degrees to at least 180 degrees, or up to 360 degrees.

Aspect 41: The assembly of aspect 40, wherein the at least one output connector port is configured to maintain electrical communication between the respective connector and the at least one output connector port while permitting the respective connector to pivot about the respective axis by an amount ranging from 0 degrees to at least 180 degrees relative to the at least one output connector port.

Aspect 42: The assembly of any one of aspects 37-41, wherein the recess of the at least one flexible projection is cylindrical.

Aspect 43: The assembly of any one of aspects 37-42, wherein the recess of each flexible projection of the at least one flexible projection has a depth from 4 mm to 10 mm, wherein the depth corresponds to a distance from the distal opening to the respective output connector port surrounded by said flexible projection.

Aspect 44: The assembly of any one of aspects 37-43, wherein the at least one flexible projection has a Shore 00 durometer from 80 to 100.

Aspect 45: The assembly of any one of aspects 37-44, wherein the at least one flexible projection has a Shore 00 durometer from 85 to 95.

Aspect 46: The assembly of any one of aspects 37-45, wherein the at least one flexible projection has a Shore 00 durometer of about 90.

Aspect 47: The assembly of any one of aspects 37-46, wherein the circuitry further comprises:

a sensor that is configured to detect a decoupling between the respective connector and the at least one output connector port; and

a current interrupter that is configured to interrupt current from the current generator at any point between the current generator and the at least one output connector port upon the sensor detecting the decoupling.

Aspect 48: The assembly of aspect 47, wherein the at least one output connector port comprises a plurality of electrical leads, wherein each electrical lead of the plurality of electrical leads is an annulus or a segment of an annulus, and wherein the sensor is configured to detect current or voltage through an outermost electrical lead of the plurality of electrical leads.

Aspect 49: The assembly of aspect 47 or aspect 48, wherein the sensor comprises at least one pressure sensor, wherein each pressure sensor is configured to detect a pressure between a respective output connector port of the at least one output connector port and the respective connector.

Aspect 50: The assembly of any one of aspects 47-49, wherein the sensor comprises at least one magnetic field strength sensor, wherein each magnetic field strength sensor is configured to detect a magnetic field strength between a respective output connector port of the at least one output connector port and the respective connector.

Aspect 51: The assembly of any one of aspects 37-50, further comprising a lock that is configured to inhibit decoupling of the respective connector from the at least one output connector port.

Aspect 52: The assembly of any one of aspects 37-51, wherein the at least one output connector port is configured to provide at least 4 electrical connections.

Aspect 53: The assembly of any one of aspects 37-52, wherein the at least one output connector port is configured to conduct at least 9 amps.

Aspect 54: The assembly of any one of aspects 37-53, wherein the at least one output connector port comprises a plurality of outlet ports.

Aspect 55: The assembly of any one of aspects 37-54, wherein the electrical input component comprises a main cable integrally coupled to the housing.

Aspect 56: The assembly of any one of aspects 37-55, wherein the electrical input component comprises a main cable connector that is configured to electrically couple to a main cable.

Aspect 57: The assembly of any one of aspects 37-56, wherein the at least one output connector port is configured to magnetically couple to the respective connector.

Aspect 58: The assembly of aspect 57, wherein the at least one output connector port comprises a magnet.

Aspect 59: The assembly of aspect 57, wherein the at least one output connector port comprises a magnetic material that is magnetizable in the presence of a magnet of the respective connector.

Aspect 60: A system comprising:

circuitry comprising:

an electrical input component that is configured to receive current from a current generator;

at least one output connector port, each output connector port of the at least one output connector port configured to magnetically couple to a respective connector such that the output connector port and the respective connector are aligned along a respective axis to maintain electrical communication between the respective connector and the at least one output connector port while permitting the respective connector to rotate and/or pivot about the respective axis relative to the at least one output connector port;

wherein the electrical input component is in electrical communication with, or configured to selectively be in electrical communication with, the at least one output connector port;

a housing comprising at least one flexible projection, wherein each flexible projection of the at least one flexible projection surrounds a respective output connector port of the at least one output connector port to form a recess defining a distal opening; and

a cable comprising the respective connector that is configured to couple to the at least one output connector port.

Aspect 61: The system of aspect 60, further comprising a transducer array comprising at least one electrode element, wherein the cable is electrically coupled to the at least one electrode element of the transducer array.

Aspect 62: The system of aspect 61, further comprising a current generator configured to cooperate with the transducer array to generate tumor treating fields.

Aspect 63: The system of aspect 62, wherein the current generator is configured to cooperate with the transducer array to the generate tumor treating fields at a frequency in a range from 50 kHz to 1 MHz.

Aspect 64: The system of any one of aspects 60-63, wherein the at least one output connector port comprises one of:

a plurality of spring pins; or

a plurality of electrical leads, wherein each electrical lead of the plurality of electrical leads is an annulus or a segment of an annulus,

wherein the respective connector of the cable comprises the other of a plurality of spring pins; or

a plurality of electrical leads, wherein each electrical lead of the plurality of electrical leads is an annulus or a segment of an annulus,

wherein an outermost electrical lead of the plurality of electrical leads and/or an outermost spring pin aligned to contact the outermost electrical lead is in electrical communication with a current interrupter that is configured to cease generation of tumor treating fields upon decoupling of a respective spring pin of the plurality of spring pins.

Aspect 65: The system of aspect 64, wherein at least one electrical leads of the plurality of electrical leads radially inward of the outermost electrical lead are configured to conduct current to generate the tumor treating fields.

Aspect 66: The system of aspect 65, wherein the connector of the cable is configured to magnetically couple to the at least one output connector port.

Aspect 67: The assembly of aspect 66, wherein the at least one output connector port comprises a magnet.

Aspect 68: The assembly of aspect 66, wherein the respective connector of the cable comprises a magnet.

Aspect 69: The system of any one of aspects 60-68, wherein the respective connector is receivable within the recess of the at least one flexible projection with a clearance of no more than 1 mm on each side.

Aspect 70: The system of any one of aspects 60-69, wherein the at least one output connector port comprises a plurality of electrical leads, wherein the plurality of electrical leads are annuluses or segments of annuluses, wherein the circuitry of the assembly further comprises:

a sensor that is configured to detect a decoupling between the respective connector and the at least one output connector port by detecting a current or voltage through an outermost electrical lead of the plurality of electrical leads; and

a current interrupter that is configured to interrupt current at any point between the current generator and the at least one output connector port upon the sensor detecting the decoupling,

wherein the respective connector of the cable comprises a plurality of spring pins, wherein each spring pin of the plurality of spring pins is configured to project outwardly by a respective maximum distance, wherein a radially outermost spring pin of the plurality of spring pins is configured to project outwardly by a first maximum distance that is less than the respective maximum distance of at least one other spring pin of the plurality of spring pins.

Aspect 71: The system of any one of aspects 60-70, wherein the respective connector comprises at least one indicator electrical connector and a plurality of conductive elements, wherein the apparatus further comprises:

at least one associated monitoring circuit configured to provide feedback related to a status of a connection between the respective connector and the at least one output connector port, the at least one associated monitoring circuit connected to the at least one indicator electrical connector configured to disconnect from the at least one output connector port at a first position relative to the at least one output connector port and wherein the first plurality of conductive elements is configured to disconnect from the at least one output connector port at a second position relative to the at least one output connector port, the second position being different from the first position.

Aspect 72: A method comprising:

generating tumor treating fields using an assembly, wherein the assembly includes:

circuitry comprising:

an electrical input component that is configured to receive current from a current generator;

at least one output connector port, each output connector port of the at least one output connector port configured to magnetically couple to a respective connector such that the output connector port and the respective connector are aligned along a respective axis to maintain electrical communication with the respective connector;

wherein the electrical input component is in electrical communication with, or configured to selectively be in electrical communication with, the at least one output connector port;

a housing comprising at least one flexible projection, wherein each flexible projection of the at least one flexible projection surrounds a respective output connector port of the at least one output connector port to form a recess defining a distal opening; and

a cable comprising the respective connector that is configured to couple to the at least one output connector port;

a transducer array comprising at least one electrode element, wherein the cable is electrically coupled to the at least one electrode element of the transducer array; and

a current generator configured to cooperate with the transducer array to generate tumor treating fields.

Aspect 73: The method of aspect 72, wherein the tumor treating fields have a frequency in a range from 50 kHz to 1 MHz.

Aspect 74: An assembly comprising:

circuitry comprising:

an electrical input component that is configured to receive current from a current generator;

at least one output connector port, each output connector port of the at least one output connector port configured to magnetically couple to a respective connector such that the output connector port and the respective connector are aligned along a respective axis to maintain electrical communication between the respective connector and the at least one output connector port;

wherein the electrical input component is in electrical communication with, or configured to selectively be in electrical communication with, the at least one output connector port.

Aspect 75: The assembly of aspect 74, wherein each output connector port of the at least one output connector port is configured to magnetically couple to a respective connector such that the output connector port and the respective connector are aligned along a respective axis to maintain electrical communication between the respective connector and the at least one output connector port while permitting the respective connector to rotate and/or pivot about the respective axis relative to the at least one output connector port.

Aspect 76: A system comprising:

an assembly that includes:

circuitry comprising:

an electrical input component that is configured to receive current from a current generator;

at least one output connector port, each output connector port of the at least one output connector port configured to magnetically couple to a respective connector such that the output connector port and the respective connector are aligned along a respective axis to maintain electrical communication between the respective connector and the at least one output connector port;

wherein the electrical input component is in electrical communication with, or configured to selectively be in electrical communication with, the at least one output connector port; and

a cable comprising the respective connector that is configured to couple to the at least one output connector port.

Aspect 77: The system of aspect 76, wherein each output connector port of the at least one output connector port is configured to magnetically couple to a respective connector such that the output connector port and the respective connector are aligned along a respective axis to maintain electrical communication between the respective connector and the at least one output connector port while permitting the respective connector to rotate and/or pivot about the respective axis relative to the at least one output connector port.

Aspect 78: The system of aspect 76 or aspect 77, further comprising:

a transducer array comprising at least one electrode element, wherein the cable is electrically coupled to the at least one electrode element of the transducer array, and

a current generator configured to cooperate with the transducer array to generate tumor treating fields having a frequency in a range from 50 kHz to 1 MHz.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim

format for a given embodiment (e.g., the given embodiment described in independent claim format) may be combined with other embodiments (described in independent claim format or dependent claim format).

What is claimed is:

1. An assembly comprising:
circuitry comprising:
an electrical input component that is configured to receive current from a current generator;
at least one output connector port, each output connector port of the at least one output connector port configured to magnetically couple to a respective connector such that the output connector port and the respective connector are aligned along a respective axis to maintain electrical communication with the respective connector;
wherein the electrical input component is in electrical communication with, or configured to selectively be in electrical communication with, the at least one output connector port;
a housing comprising at least one flexible projection, wherein each flexible projection of the at least one flexible projection surrounds a respective output connector port of the at least one output connector port to form a recess defining a distal opening.

2. The assembly of claim 1, wherein each output connector port of the at least one output connector port comprises a magnet.

3. The assembly of claim 1, wherein each output connector port of the at least one output connector port comprises a magnetic material that is magnetizable in the presence of a magnet of the respective connector.

4. The assembly of claim 1, wherein each output connector port of the at least one output connector port comprises one of:
a plurality of spring pins; or
a plurality of electrical leads, wherein each electrical lead of the plurality of electrical leads is an annulus or a segment of an annulus.

5. The assembly of claim 4, wherein each output connector port of the at least one output connector port comprises a plurality of electrical leads, wherein each electrical lead of the plurality of electrical leads is an annulus or a segment of an annulus.

6. The assembly of claim 1, wherein each output connector port of the at least one output connector port is configured to maintain electrical communication between the respective connector and said output connector port while permitting the respective connector to rotate about the respective axis relative to said output connector port by an amount ranging from 0 degrees to at least 180 degrees, or up to 360 degrees.

7. The assembly of claim 1, wherein each output connector port of the at least one output connector port is configured to maintain electrical communication between the respective connector and said output connector port while permitting the respective connector to pivot about the respective axis by an amount ranging from 0 degrees to at least 180 degrees relative to said output connector port.

8. The assembly of claim 1, wherein the recess of each flexible projection of the at least one flexible projection is cylindrical.

9. The assembly of claim 1, wherein the recess of each flexible projection of the at least one flexible projection has a depth from 3 mm to 1 cm, wherein the depth corresponds to a distance from the distal opening to the respective output connector port surrounded by said flexible projection.

10. The assembly of claim 1, wherein each flexible projection of the at least one flexible projection has a Shore 00 durometer from 80 to 100.

11. The assembly of claim 1, wherein the circuitry further comprises:
a sensor that is configured to detect a decoupling between the respective connector and the at least one output connector port; and
a current interrupter that is configured to interrupt current between the current generator and the at least one output connector port upon the sensor detecting the decoupling.

12. The assembly of claim 11, wherein the at least one output connector port comprises a plurality of electrical leads, wherein each electrical lead of the plurality of electrical leads is an annulus or a segment of an annulus, and wherein the sensor is configured to detect current or voltage through an outermost electrical lead of the plurality of electrical leads.

13. The assembly of claim 11, wherein the sensor comprises at least one pressure sensor, wherein each pressure sensor of the at least one pressure sensor is configured to detect a pressure between a respective output connector port of the at least one output connector port and the respective connector.

14. The assembly of claim 11, wherein the sensor comprises at least one magnetic field strength sensor, wherein each magnetic field strength sensor of the at least one magnetic field strength sensor is configured to detect a magnetic field strength between a respective output connector port of the at least one output connector port and the respective connector.

15. The assembly of claim 1, wherein the electrical input component comprises a main cable integrally coupled to the housing.

16. The assembly of claim 1, wherein the electrical input component comprises a main cable connector that is configured to electrically couple to a main cable.

17. A system comprising:
an assembly that includes:
circuitry comprising:
an electrical input component that is configured to receive current from a current generator;
at least one output connector port, wherein each output connector port of the at least one output connector port is configured to magnetically couple to a respective connector such that the output connector port and the respective connector are aligned along a respective axis to maintain electrical communication with the respective connector port;
wherein the electrical input component is in electrical communication with, or configured to selectively be in electrical communication with, the at least one output connector port;
a housing comprising at least one flexible projection, wherein each flexible projection of the at least one flexible projection surrounds a respective output connector port of the at least one output connector port to form a recess defining a distal opening; and
a cable comprising the respective connector that is configured to couple to the at least one output connector port.

18. The system of claim 17, further comprising a transducer array comprising at least one electrode element, wherein the cable is electrically coupled to the at least one electrode element of the transducer array.

19. The system of claim 17, further comprising a current generator configured to cooperate with the transducer array to generate tumor treating fields having a frequency in a range from 50 kHz to 1 MHz.

20. A method comprising:
generating tumor treating fields using an assembly, wherein the assembly includes:
circuitry comprising:
an electrical input component that is configured to receive current from a current generator;
at least one output connector port, each output connector port of the at least one output connector port configured to magnetically couple to a respective connector such that the output connector port and the respective connector are aligned along a respective axis to maintain electrical communication with the respective connector;
wherein the electrical input component is in electrical communication with, or configured to selectively be in electrical communication with, the at least one output connector port;
a housing comprising at least one flexible projection, wherein each flexible projection of the at least one flexible projection surrounds a respective output connector port of the at least one output connector port to form a recess defining a distal opening; and
a cable comprising the respective connector that is configured to couple to the at least one output connector port;
a transducer array comprising at least one electrode element, wherein the cable is electrically coupled to the at least one electrode element of the transducer array; and
a current generator configured to cooperate with the transducer array to generate tumor treating fields.
